(19) United States
(12) Patent Application Publication
(10) Pub. No.: US 2008/0144306 A1
(43) Pub. Date: Jun. 19, 2008

(54) Surgical headlamp

(76) Inventor: David Medinis, (US)

Correspondence Address: EDWIN W. OLDHAM, 400 PARK SHORE DR. # 200, NAPLES, FL, 34103, US

(21) Appl. No.: 11/800,512

(22) Filed: May 7, 2007

Related U.S. Application Data
(63) Continuation-in-part of application No. 11/638,940, filed on Dec. 14, 2006.

(51) Int. Cl. A61B 1/06

(52) U.S. Cl. 362/105

(57) ABSTRACT

The invention comprises a flexible tubing which is adapted to carry a plurality of electrical conductor lines and have at least one passage for passing fluids through and wherein there is a mounting connector that connects the electrical lines to the desired use of it and also a connection to allow the fluids used in the flexible tubing to be connected to the area of use. The connection is made through a friction-force fit and that achieves both the electrical connection and the fluid connection.

This new invention is a continuation in part of application Ser. No. 11/638,940 filed Dec. 14, 2006, entitled Surgical Headlamp. The present invention pertains to lights that can be worn on a user's head to provide illumination in an area of work, and, more particularly, to a headlight for surgeons, dentists or other medical personnel or craftsmen. The light uses two independent lamp housings working cooperatively to converge light beams at a predetermined distance from the LED light sources.

### I. BACKGROUND OF THE INVENTION

A. Field of Invention

It is essential in certain medical procedures that the physician, surgeon, or Dentist has his or her hands free for manipulating various surgical diagnostic or therapeutic instruments. At the same time, the particular part of the patient's body that the physician or surgeon is treating must be adequately illuminated. For these purposes, doctors and surgeons have heretofore utilized surgical head lights, some of which require the user to remain attached by via fiber optics to a free-standing light source, and/or to a power outlet or an energy source.

Battery powered head-mounted lamps utilizing an incandescent lamp as a light have also been used. Typically, the high power consumption, relatively low light output, high weight, and short battery life of each device of the prior art have made their use difficult, uncomfortable, or otherwise unsatisfactory.

Even with such configurations, however, the amount of light illuminating upon the work area can be inadequate. There have been attempts to increase the light pinching upon the work area by utilizing xenon or halide lamps, which require high power and have a relatively short bulb life and generate substantial amounts of heat.

Some of the problems associated with such xenon and halide lights may be overcome by utilizing light-emitting diodes (LEDs).

Some prior art apparatus have used relatively high-powered LEDs to generate sufficient light output. Such LEDs typically generate so much heat that a heat sink is required; heretofore the prior art has not been able to satisfy the repairment of a heat sink or cooling system for LED light generation.

Several attempts to solve the problems described herein above have been made in the prior art. For example, published U.S. Pat. No. 6,055,444, published Jun. 2, 2005, and issued as a patent on Oct. 18, 2005 for Surgical Headlight by Suhil Gupta, teaches a head-mounted lamp assembly with at least two LEDs mounted side by side and focused utilizing a rear reflector. Such rear reflector use greatly diminishes the efficiency of the projection of the light generated by the LEDs and thus is unsatisfactory for providing a high intensity, focused light beam of the apparatus of the present invention.

U.S. Pat. No. 7,108,400 entitled Light Source Unit and Projector, by inventor Shuhei Yamada and Takeshi Seto, teaches the use of a LED light source for illumination of high ruminants, which includes a cooling system for the illumination of high luminants, which generates substantial heat. This design utilizes two liquid heat source absorbers and is very complex and is much more difficult to implement than that cooling system taught by the present invention.

U.S. published Patent Application No. 2005/0243539 teaches a cooled light emitting apparatus comprising a light source including a close-packed array of light emitting diodes and a cooling system for cooling the light source. The cooling system is a thermoelectric cooling device in the form of a peltier device connected by a heat spreader to the light source and a heat exchange system for removing heat from the peltier device. The heat exchange system utilizes a liquid coolant to cool the peltier device in this instance the invention utilizes a heat pipe configuration or arrangement, and this is far less satisfactory than the liquid cooling system taught by the present invention.

### II SUMMARY OF THE INVENTION

In accordance with the present invention, there is provided a surgical headlight that overcomes the problems of head-mounted surgical headlamps of the prior art. A plurality of LED's are mounted in a circular arrangement on a printed circuit board that includes a metal substrate heat sink associated therewith that is in contact with a coolant chamber and a belt-mounted cooling liquid is pumped through the cooling chamber to control the heat output from the LED array mounted under and focused by a focus lens that is a single lens that focuses all of the diodes into a coherent focused pattern at a particular point of desired focus and intensity.

It is, therefore, an object of the invention to provide a head-mounted LED based headlamp for use by surgeons or other medical personnel, etc. It is another object of the invention to provide a head-mounted LED based headlamp comprised of a pair of individual lamp housings, each incorporating a plurality of LED cells and each unit mounted with associated with a focus lens that provides a 6″ focus angle for an effective 200 mm circle of light at 440 mm.

It is a further object of the invention to provide a head-mounted LED-based headlamp powered by rechargeable batteries.

It is a further object of the invention to provide a head-mounted LED-based headlamp which has a bulb life of at least 50 times that of a xenon/halide bulb while operating at a less than ⅙ the wattage requirement of such xenon/halide bulbs.

It is a further object of the invention to provide a head-mounted LED-based headlamp which is very reasonable in cost and provides a significant solid-state semi-coherent light for passage through a focus lens at a 6% ocus angle for an effective 200 mm circle of light at 440 mm.

### III. BRIEF DESCRIPTION OF THE DRAWINGS

TA complete understanding of the present invention may be obtained by reference to the accompanying drawings, when considered in conjunction with the subsequent detailed description, in which:

FIG. 1 is a side view in schematic form of the headlight assembly system of the invention showing the head mount strap, the headlight itself and the remotely located cooling and battery powered system assembly;

FIG. 2 is a top schematic view of the headlight assembly utilized as one of the two assemblies associated with the headlamp of the invention and showing the focus lenses, each individually hexagonal in shape, and arranged into a hexagonal circular shape with the six lenses toughing on the flat outer sides, as shown;

FIG. 3 is a side cross-sectional elevation, taken on line 3-3 of FIG. 2 of the three LEDs associated with one side of the headlamp and showing the construction the focusing lenses and the heat sink associated therewith as well as the cooling chamber;

FIG. 4 is a cross-sectional elevation showing of one of the focusing lenses showing the relationship between the cone of light emission from the LED and the resultant condensing of that emission into a focused cone of light achieved by the focusing lens;

FIG. 5 is a schematic perspective showing of the six LEDs in hexagonal array, and each pointed at the specific focal point desired;

FIG. 6 is a perspective view of the headlamp of FIG. 1 showing two headlamps in side-by-side relationship: and.

FIG. 7 is an electrical diagram of the headlight assembly showing the battery, the LEDs, the pump and an on-off switch.

FIG. 8 is an exploded cross-sectional view of the electrical and fluid systems associated with the tubing that connects the power pack to the headlamp.

For purposes of brevity and clarity, components and elements of the apparatus of this invention will bear the same designations or numbering throughout the Figures.

### IV. DETAILED DESCRIPTION OF THE PREFERRED EMBODIMENT

The present invention provides a surgical headlight which may be used by surgeons, physicians, dentists, etc., who require a reliable, portable, high density, battery-power light source. For the sake of brevity, the term surgeon is used hereinafter to refer to any user of the head-mounted headlamp of the invention.

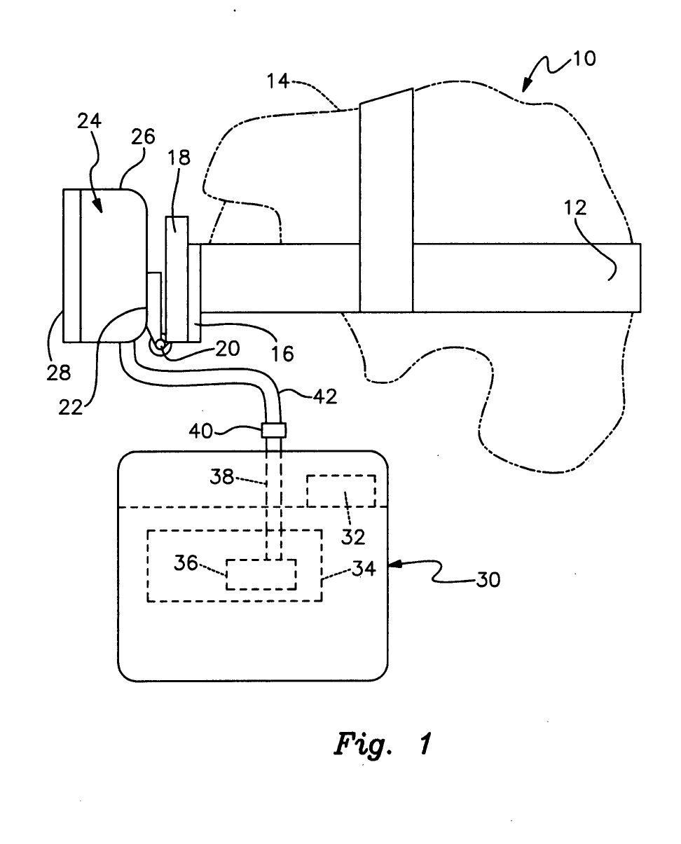

Referring first to FIG. 1 of the drawings, 10 indicates generally the head-mounted headlamp of the invention which comprises a basic band 12 to go around the head and forehead of the user, and this band 12 is normally adjustable to be able to therefore fit the size of the wearer of the unit. A top strap 14 is shown, which goes over the top of the head of the user and stabilized the whole assembly to fit comfortably and securely onto the user's head, in much the typical way of any of these devices.

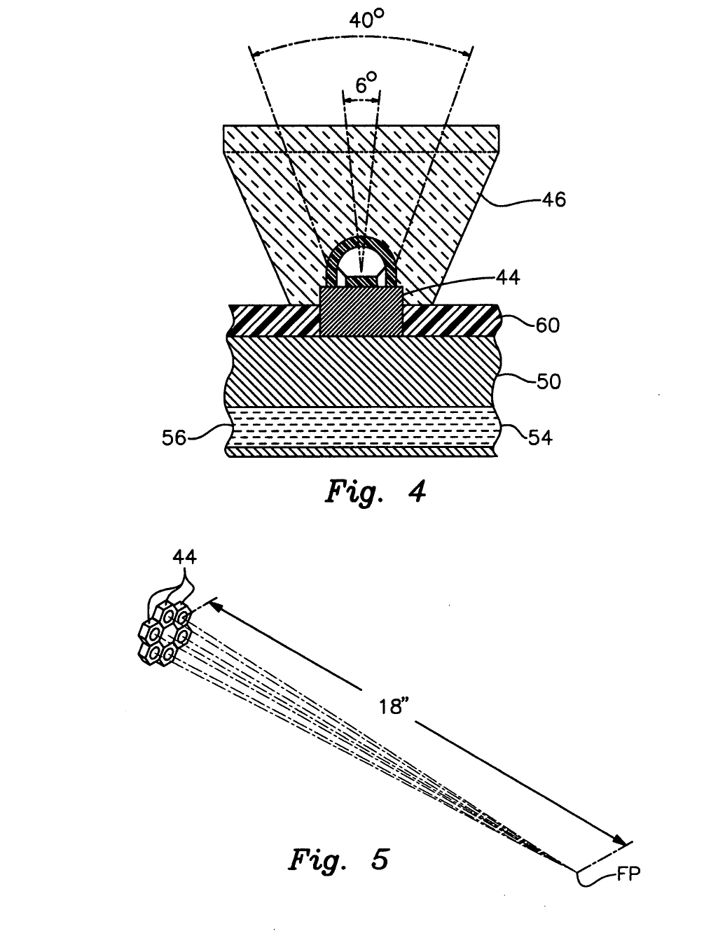

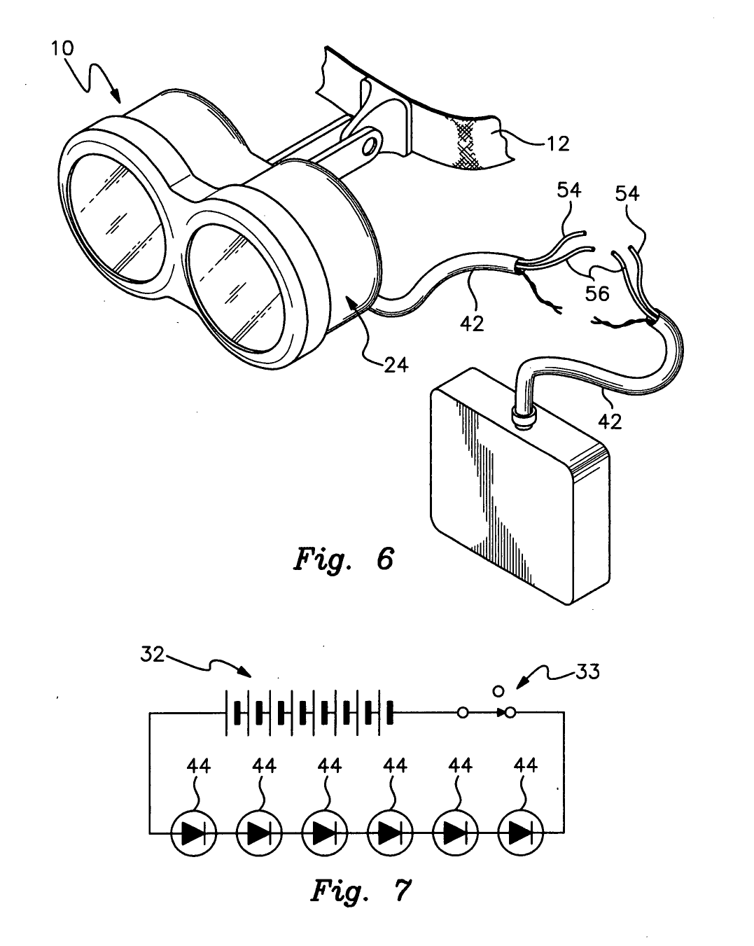

The band 12 mounts then to a mounting plate 16 which attaches itself to a further forehead plate 18 and plate 18 operates through a ratcheting swivel 20 to provide an actual mounting plate 22 which carries the improved head-mounted headlamp 24 of the invention. The basic headlamp 24 is composed of two separate lamps 24, as best seen in FIG. 6. The basic headlamp is comprised of a rearward housing 26 which carries the actual LED configuration and cooling, to be described later, and a front protective cover for the focusing lenses as described in conjunction with FIG. 4.

The auxiliary package is indicated generally by 30 and this is remotely mounted, normally on the waist or the back, through a belt arrangement typically suitable for the user. This auxiliary package incorporates a LED power supply 32 is a conventional rechargeable battery typically used for this type of system and is conveniently located for replacement or recharging in the box assembly 30. The box 30 also incorporates a coolant reservoir 34, again shown in dotted line, and coolant reservoir incorporates a coolant pump 36, again shown in dotted line and the pump 36 transmits coolant through a coolant tube 38, also indicated by dotted line within the box 30 and then there is a quick connect or disconnect unit 40 located between the flexible tubing that constitutes the tubing 42 that sends the coolant fluid up and into its appropriate use for cooling in the headlamp assembly 24.

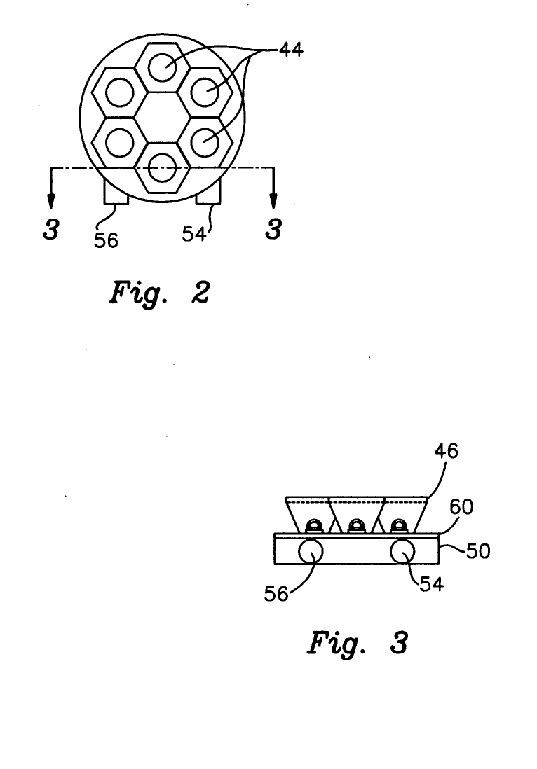

Referring now to the headlamp assembly 24 shown in FIG. 2 of the drawings, the headlamp includes a plurality of LED's indicated by 44. The LEDs are mounted in a hexagonally-shaped focusing lens 28 that comprises a plurality of independent lenses 46 associated with each LED. The lens 28 is preferably a solid polymer molding to incorporate six individual lenses 46, all physically connected to a central hexagon piece 47, all as one overall piece by a separate mold that locates the lenses as shown in FIG. 5 so as to focus at a point approximately 18 inches from the lenses 46. This will be explained in more detail with respect to the drawings shown in FIGS. 3 and 4.

The LEDs 44 are wired in series to the power supply, as shown in FIG. 7, with an with an on-off switch 33, and this switch 33 also simultaneously turns on the fluid pump 36. The solid-state, semi-coherent light passes through the focusing lens 46 at a 60 focus angle for an effective 200 mm circle of light at 440 mm. The alignment of the LEDs 44 and the focusing lenses 46 is best seen in FIG. 4 where it shows that the LEDs actually put out an approximately 80° cone of semi-coherent light, which is condensed into a much narrower approximately 6° of conical light with each respective LED focused at the same point, as shown in FIG. 5. These actual distances and relationships can be varied depending upon the particular focus characteristics of the focus lenses 46. It is important to the proper functioning of the LEDs that the LEDs draw no more than about 50 watts of battery power and preferable the battery 32 will be between 12 and 24 volts to produce a load of 1 amp to power each of the 12 LEDs in the two-unit headlamp 24.

Referring again to FIG. 7 of the drawing, the battery 32 is connected through the closing of switch 33 to provide 1 amp in power to the LED array. The LEDs are each preferable of 5 watt power output. The LEDs 44 working together form a light engine for each of the headlamps 24 of FIG. 6. The LEDs 44 are activated and emit bright semi-coherent light through the focus lenses 46 now probably best seen in both FIGS. 3 and 4. This light is then emitted to the focus lenses 46, as best seen in FIG. 4. It is important to note, also, that the semi-coherent light emitted by the LEDs is in a lamberton distribution.

The heat generated from the LEDs is drawn out through an aluminum heat sink layer 50. With the coolant flowing in the coolant chamber 52, excess heat is carried away from the assembly via the coolant output 54 and coolant input 56, which brings the coolant into the chamber 52. Coolant input 56 and output 54 are connected to a coolant reservoir and pump with flexible tubing, as already described with respect to FIG. 1. The coolant can be water, and it has been found that the flow rate of 400 ml per minutes works very well.

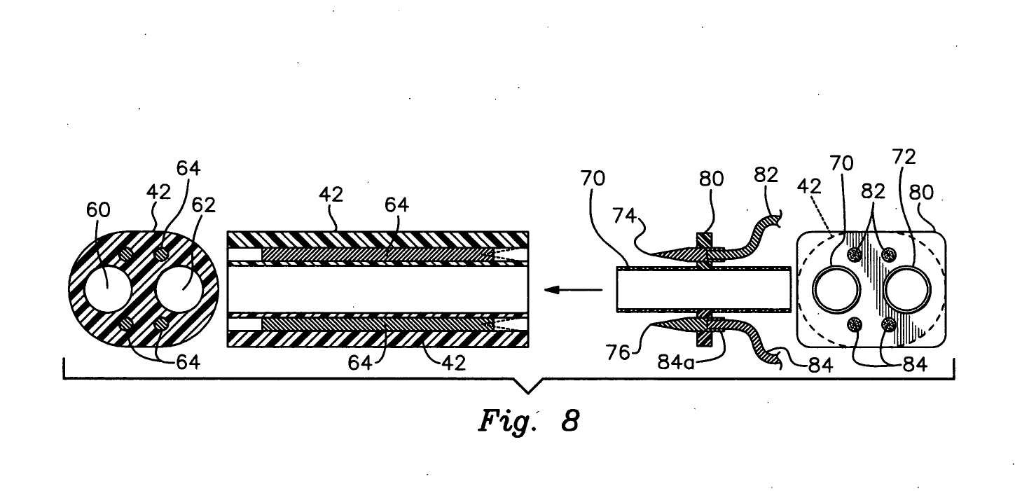

Referring now specifically to FIG. 8, the structure and function of the cooling and electrical tubing 42 will be described in detail. The tubing 42 is preferably extruded and the preferable material is silicone, or other equivalent substitutes which will provide both an insulation characteristic and an ability to extrude around metal wires and to form simultaneously the holes for passing the fluid. The holes are indicated by numerals 50, 60 and 62, and the wires are indicated generally by numeral 64. The tubing 42 acts an electrical insulator from the wires 64. The wires 64 are preferably multi-stranded twisted copper and as stated above are formed simultaneously in the extrusion. The fluid carrying openings 60 and 62 are again formed by a standard extrusion process for the tubing 42. Preferably, the wires 64 will be spaced sufficient distance from the openings 60 and 62 so that there will not be any possible interconnection and leakage of either electricity or fluid in the tubing 42. It should be understood that the front portion of the FIG. 8 is the cross sectional configuration of the tubing 42 in the longitudinal direction and the cross sectional illustration immediately to the right is a cross sectional configuration of the tubing 42 in the lengthwise direction.

The connector portion of the tubing 42 is shown on the right side of FIG. 8 and indicates a connector tubing 70 and 72, which is designed for a snug fit of the interior of the openings 60 and 62 respectively on the left side of FIG. 8. The remaining configuration of the connector incorporates sharply pointed pins identified 74 and 76 that are designed to again fit snugly into the openings shown at the right side of the tubing 42 of FIG. 8 and are shown in dotted line position in their fully inserted position to achieve the electrical contact necessary to the wires 64. The pins 74 and 76 are then connected to a flexible wire identified 82 and 84 that are attached to suitable connecting rings 82a and 84a as shown to the right side of FIG. 8. A similar type connector is shown on the right-hand side of FIG. 8 and will be utilized on the left-hand end of the tubing 42 shown to the left of FIG. 8 as necessary to connect it to the headlamp, for example, different lengths of tubing 42 can be connected by multiple connectors to achieve the desires length of the tubing 42 necessary for the particular job.

Since other modifications and changes vary to fit particular operating requirements and environments it will be apparent to those skilled in the art. Invention is not considered limited to the examples chosen for purposes of disclosure and covers all changes and modifications which do not constitute departures from the true spirit and scope of the invention.

Having thus described the invention, what is desired to be protected by letters patent is presented in the subsequently appended claims.

What is claimed is:

1. A tubing adapted to carry both a fluid stream and an electrical signal that is characterized by:
An extruded tubing made from an insulating material and also being highly flexible without breaking or cracking, the extrusion containing at least one open passage for a flow of fluid there-through,
At least one electrical conductor extruded simultaneously into the extruded essentially through the full length of the tubing, and
The means to connect the lengths of tubing to a fluid supply and an electrical connection.

2. A tubing according to claim 1 where the connection is at least one pin mounted to a pull flange arranged to fit in relationship into the tubing and make electrical contact with the extruded wire, and which further includes an open tubular connector to the pull flange adapted to make frictional snug contact with the inside diameter of the openings in the tubing.

3. Tubing according to claim 2 where the pin is sharply pointed and tapers down to a diameter that will snuggly fit the base of the pin into the tubing, and 4, Tubing according to claim 1 which is arranged to carry two fluid passageways and four electrical conductors.
